SEQUENCE LISTING (4 sequences)

SEQ ID NO: 1 - protein, 207 aa, Thaumatococcus daniellii
ATFEIVNRCSYTVWAAASKGDAALDAGGRQLNSGESWTINVEPGTNGGKIWARTDCYFDDSGSGICKTGDCGGLLRCKRFGRPPTTLAEFSLNQYGKDYIDISNIKGFNVPMNFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTA

SEQ ID NO: 2 - protein, 207 aa, Thaumatococcus daniellii
ATFEIVNRCSYTVWAAASKGDAALDAGGRQLNSGESWTINVEPGTKGGKIWARTDCYFDDSGRGICRTGDCGGLLQCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTA

SEQ ID NO: 3 - protein, 207 aa, Thaumatococcus daniellii
ATFEIVNRCSYTVWAAASKGDAALDAGGRQLNSGESWTINVEPGTNGGKIWARTDCYFDDSGSGICKTGDCGGLLRCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTA

SEQ ID NO: 4 - protein, 207 aa, Thaumatococcus daniellii
ATFEIVNRCSYTVWAAASKGDAALDAGGRQLNSGESWTINVEPGTKGGKIWARTDCYFDDSGSGICKTGDCGGLLRCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTA

(19) United States
(12) Patent Application Publication
(10) Pub. No.: US 2024/0373891 A1
(43) Pub. Date: Nov. 14, 2024

(54) SWEETENER BLEND COMPRISING THAUMATIN AND MOGROSIDES

(71) Applicant: NOMAD BIOSCIENCE GMBH, München (DE)

(72) Inventors: Anett Stephan, Halle (Saale) (DE); Anatoli Giritch, Halle (Saale) (DE); Yuri Gleba, Berlin (DE)

(73) Assignee: NOMAD BIOSCIENCE GMBH, München (DE)

(21) Appl. No.: 18/683,563

(22) PCT Filed: Aug. 22, 2022
(86) PCT No.: PCT/EP2022/073337

§ 371 Date: Feb. 14, 2024

Related U.S. Application Data
(60) Provisional application No. 63/235,260, filed on Aug. 20, 2021.

(51) Int. Cl. A23L 27/30

(52) U.S. Cl. CPC A23L 27/31; A23L 27/36

(57) ABSTRACT

The invention provides a sweetener or flavor modifying composition comprising Thaumatin of greater than 90% purity and at least one Mogroside.

### FIELD OF THE INVENTION

The present invention relates to a low-calorie sweetener composition having sweetness synergy and improved organoleptic properties comprising Thaumatin, such as Thaumatin II, and one or more of Mogroside including Mog IIIe, Siamenoside I, Mog IV, Mog V, Mog VI and/or Siratose. The present invention also relates to food and beverage products comprising said sweetener composition. The invention also relates to a kit-of-parts comprising Thaumatin, such as Thaumatin II, and one or more of Mogroside including Mog IIIe, Siamenoside I, Mog IV, Mog V, Mog VI and/or Siratose.

### BACKGROUND

Most food and beverage products contain nutritive sweeteners such as sucrose (generally referred to as ‘sugar’ or ‘table sugar’), glucose, fructose, corn syrup, high fructose corn syrup and the like, or high intensity sweeteners such as aspartame, sucralose, acesulfame K, saccharin, cyclamates, steviol glycosides, monk fruit extract and the like. Such sweeteners supply sweetness and a favorable sensory response, for example in terms of flavor balance, quality of sweetness, lack of bitterness and off taste, desirable temporal profile and desirable mouthfeel.

Despite the preferred taste and functional properties provided by nutritive sweeteners like sugar, excess intake of full calorie sweeteners has long been associated with an increase in diet-related health issues, such as obesity, heart disease, metabolic disorders and dental problems. This worrying trend has caused consumers to become increasingly aware of the importance of adopting a healthier lifestyle and reducing the level of nutritive sweeteners in their diet.

Recently there has been a substantial increase in consumer products utilizing replacements for nutritive sweeteners, with a particular focus on the development of low or zero-calorie sweeteners of natural origin. In fact, in North America, the number of new product launches utilizing natural high intensity low calorie sweeteners has increased, outpacing artificially sweetened new product launches, while nutritive sweetened product launches have slowly declined.

An ideal replacement for a nutritive or artificial sweetener is a sweetener that has desirable taste characteristics with fewer calories, can be sustainably produced, has clearly understood metabolism and history of safe use, and is cost effective. Aiming to meet this growing need, the market has been flooded with possible candidates to replace conventional nutritive sweeteners. Unfortunately, however, many of the low or zero calorie replacements offered on the market lack one or all of the necessary characteristics, and often exhibit bitterness or off-taste. Therefore, many of the proposed sweeteners are not an ideal replacement for nutritive sweeteners.

Thaumatin, a protein found in Katemfe fruit, has been well known as a sweetener and flavor modifier. However, as Katemfe fruit is not readily cultivated, sustainability of the thaumatin market is poor and cost is high. Additionally, thaumatin from Katemfe fruit is a mixture of thaumatin proteins, some of which have better organoleptic properties such as thaumatin II, and some which do not have preferred organoleptic properties. This is further complicated as thaumatin from katemfe fruit is a natural product and the ratios of thaumatins are variable in addition to the quality. Furthermore, while thaumatin alone has good taste properties when providing a sweetness equivalent to a 5% solution of sucrose, at higher sweetening concentrations it suffers from off taste properties such as bitter, metallic, or medicinal notes. Recent advances in technology allow the production of thaumatin through transient expression of this protein in well-known agricultural commodity crops which has eliminated the variability, mixed nature, and reduced the cost in use resulting in a readily available source of thaumatin II, which has improved organoleptic properties vs. mixed thaumatins. However, at higher sweetening concentrations, thaumatin II from commodity crop expression still suffers from off taste properties, for example extended sweetness lingering.

Monk Fruit or the fruit from Siraitia grosvenorii also known as luohan guo or LHG contains a class of sweet molecules known as mogrosides that are approximately 250 times as sweet as sucrose at the threshold level of sweetness detection. This fruit has been used for centuries in China and recently has come into the spot light in the western world as a natural sweetener and flavor modifier. The natural extract is typically a mixture of mogrosides including Mog I, Mog, II, Mog III, Mog IIIe, Mog IV, Mog V, Mog VI, and Siamenoside I. Additionally some mogrosides containing alpha glycosidic bonds have been isolated and used as sweeteners and flavor modifiers such as Siratose (see structural formula below). While the promise of monk fruit as a sweetener is high, it suffers from high cost in use and off tastes such as long sweet linger, musty, melon flavors, licorice, medicinal, and moderate bitterness when used at higher levels of sweetness. While there is substantial effort and some success in the production or improvement of mogrosides or monk fruit sweetener via biotechnology, largely cost in use and the taste properties at high levels have limited the growth of this ingredient family.

Therefore, there is a need to provide an improved replacement of natural origin for nutritive sweeteners that has low or zero-calories and is without limitations in use, but which also has preferred taste characteristics, is sustainable, has high solubility, and good cost in use.

### SUMMARY OF THE INVENTION

The present invention seeks to provide a solution to the above-mentioned problems by providing a sweetener composition of natural origin, acceptable cost in use, preferred organoleptic properties, history of safe use, and low-calorie content. The present invention also seeks to provide a sweetener composition having sweetness synergy, a reduction in off tastes or off flavors, desirable temporal profile, when compared with other high intensity sweeteners of natural origin.

The first aspect of the present invention provides a sweetener or flavor modifying composition comprising purified Thaumatin, such as Thaumatin II, of greater than 90% purity and one or more Mogroside including Mog IIIe, Mog IV, Mog V, Mog VI, Siamenoside I, or Siratose. In a second aspect, the invention provides a sweetener or flavor modifying composition comprising Thaumatin, preferably Thaumatin II, and at least one Mogroside. Similarly as in the first aspect, the Mogroside may be selected from Mog IIIe, Mog IV, Mog V, Mog VI, Siamenoside I, Siratose, and mixtures of one or more thereof. In both aspects, Mogroside V and Siamenoside I or a mixture thereof are preferred herein. In one embodiment, the Mogroside is Mogroside V. In another embodiment, the Mogroside is Siamenoside I. In both aspects, the Thaumatin may be selected from Thaumatin I, Thaumatin

II, Thaumatin A, and Thaumatin B, wherein Thaumatin II is preferred. In a preferred embodiment, the Thaumatin is Thaumatin II and the Mogroside is Mogroside V and/or Siamenoside I, preferably Mogroside V or Siamenoside I.

In one embodiment of the present invention, the sweetener or flavor modifying composition comprises Thaumatin II in an amount of about 1% to about 10%, and one or more Mogroside including Mog IIIe, Mog IV, Mog V, Mog VI, Siamenoside I, or Siratose in an amount of about 90% to about 99% by weight relative to the total weight of the thaumatin and mogroside portion of the composition.

In another embodiment, the sweetener or flavor modifying composition comprises Thaumatin, such as Thaumatin II, in an amount of about 3% and one or more Mogroside including Mog IIIe, Mog IV, Mog V, Mog VI, Siamenoside I, or Siratose in an amount of about 97% by weight relative to the total weight of the thaumatin and mogroside portion of the composition.

In another embodiment, the sweetener or flavor modifying composition comprises Thaumatin, such as Thaumatin II, in an amount of about 7% by weight and one or more Mogroside including Mog IIIe, Mog IV, Mog V, Mog VI, Siamenoside I, or Siratose in an amount of about 93% by weight relative to the total weight of Thaumatin and Mogroside portion of the sweetening or flavor modifying composition.

In some embodiments, the thaumatin portion, preferably the Thaumatin II portion, utilized in the composition is greater than 90% purity relative to the weight of the total thaumatin portion of the composition.

In some embodiments, the sweetener composition further comprises another sweet tasting additive, a stabilizing or solubilizing solvent, a bulking agent, a flavoring agent, and/or a stabilizer.

A further aspect of the present invention provides a food or beverage product comprising a sweetener composition of the invention.

In another embodiment, the sweetener or flavor modifying composition in used in the resulting food or beverage product comprises Thaumatin, such Thaumatin II, at a concentration in the food or beverage product at about 0.2 ppm and 10 ppm, preferably at about 0.2 ppm to 10 ppm, and one or more Mogroside including Mog IIIe, Mog IV, Mog V, Mog VI, Siamenoside I, or Siratose at a concentration in the food or beverage product in at about 20 ppm to 750 ppm.

A further aspect of the present invention provides a table-top sweetener comprising a sweetener composition of the invention.

Further aspects of the present invention provide a bulking agent comprising a sweetener composition according to the invention; a coating agent comprising a sweetener composition according to the invention; a cosmetic product comprising a sweetener composition according to the invention; a pharmaceutical product comprising a sweetener composition according to the invention; a nutritional product comprising a sweetener composition according to the invention; and a sports product comprising a sweetener composition according to the invention.

Another aspect of the present invention provides the use of a sweetener composition according to the present invention in a food product, a beverage product, a pharmaceutical product, a nutritional product, a sports product, or a cosmetic product. Another aspect of the present invention provides the use of the sweetener composition according to the present invention as a bulking agent. Another aspect of the present invention provides the use of the sweetener composition according to the present invention as a coating agent.

In another aspect, the invention provides a kit-of-parts comprising, as a first part, Thaumatin, preferably Thaumatin II, or a composition comprising said Thaumatin and, as a second part, at least one Mogroside or a composition comprising at least one Mogroside. The Mogroside and preferred Mogrosides are as indicated above. The mass ratios of Thaumatin, preferably Thaumatin II, in the first part and the Mogroside(s) in the second part may be as given above for the sweetener or flavor modifying composition.

### BRIEF DESCRIPTION OF THE FIGURE

FIG. 1. The graph illustrates the total mean sweet perception for Thaumatin II, Mogroside V, and the combination thereof. 5% sucrose solution in water was used as a sweetness control.

FIG. 2. Structure of Mogroside.

### DETAILED DESCRIPTION

The present invention is based upon the discovery that Thaumatin, notably Thaumatin II, surprisingly has unique characteristics when highly purified from other thaumatins and combined with Mogroside including Mog IIIe, Mog IV, Mog V, Mog VI, Siamenoside I, or Siratose. Thaumatin exists naturally as several isoforms in Katemfe fruit (Thaumatococcus daniellii). Thus, the term Thaumatin is generic and relates to any isoform or mixture of two or more isoforms of Thaumatin. These isoforms have different levels of sweetness, sweet linger and off taste. Thaumatin II is the isoform which has the best quality of sweetness with the fewest negative organoleptic properties. Therefore, Thaumatin II the preferred Thaumatin in the present invention. However, it is difficult to separate or enrich Thaumatin II from the natural Katemfe fruit extract mixture of thaumatin isoforms. However, in the present invention, Thaumatin II with, preferably, a purity of greater than 90% by weight has been utilized as isolated by transient recombinant expression in sustainable agricultural commodity crops. This has the dual benefit of only producing the best isoform Thaumatin II and doing so in a sustainable manner. Transient recombinant expression of Thaumatins, such as Thaumatin II, is known in the art and is, for example, described in WO 2022/012926 A1.

Surprisingly, when Thaumatin, notably Thaumatin II, preferably of purity greater than 90%, is combined with one or more Mogroside including Mog IIIe, Mog IV, Mog V, Mog VI, Siamenoside I, or Siratose, the composition has an improved quality of taste and a higher relative sweetness or sweetness synergy. That is to say, the relative sweetness of the sweetener composition is greater than the sweetness calculated from the individual components of the composition and furthermore the negative taste attributes of the individual components are reduced in the final composition, in particular, with regard to the bitter taste and/or undesirable temporal profile that may be associated with the individual components. In addition, due to each of the components contributing near zero calories in use, the sweetener composition is zero or low calorie. Furthermore, as a consequence of the sweetness synergy exhibited by the composition, the amount of the composition required to provide a given level of sweetness is less than would be expected in the absence of synergy, thereby allowing a further reduction in both cost and calories. Thus, the sweetener composition of the present invention provides enhanced sweetness, improves the balance of flavor by reducing off-taste, and provides a more desirable temporal profile, while at the same time allowing a significant reduction in calories and cost compared to a sweet-equivalent amount of a conventional nutritive sweetener or other natural high intensity sweeteners used individually.

Using the sweetener or flavor modifying composition of the present invention allows delivery of an increased sweetness in food or beverage products when compared to the individual components used separately. This enhanced sweetness means that a smaller amount of sweetener can be used in these products, and therefore the cost in use is reduced. Additionally, the sweetening or flavor modifying composition provides a temporal and taste profile that more closely mimics nutritive sweeteners, while maintaining other benefits such as better overall acceptability, mouthfeel, reduced off-taste, desirable temporal profile, while being cost effective.

In general terms, the present invention relates to a sweetener composition comprising the sweeteners Thaumatin, preferably Thaumatin II, and one or more Mogroside including Mog IIIe, Mog IV, Mog V, Mog VI, Siamenoside I, or Siratose.
Herein, the generic term “Thaumatin” refers to any one or more of Thaumatin isoforms I, II, A, and B. The term “Thaumatin I” refers to an oligopeptide or protein of the following amino acid sequence (SEQ ID NO: 1):
ATFEIVNRCSYTVWAAASKGDAALDAGGRQLNSGESWTINVEPGTNGGKIWARTDCYFDDSGSGICKTGDCGGLLRCKRFGR PPTTLAEFSLNQYGKDYIDISNIKGFNVPMNFSPTTRGCRGVRCAADIVGQCPAKLKAPGGGCNDACTVFQTSEYCCTTGKCGP TEYSRFFKRLCPDAFSYVLDKPTTVTCPGSSNYRVTFCPTA
The term “Thaumatin II” refers to an oligopeptide or protein of the following amino acid sequence (SEQ ID NO: 2):
ATFEIVNRCSYTVWAAASKGDAALDAGGRQLNSGESWTINVEPGTKGGKIWARTDCYFDDSGRGICRTGDCGGLLQCKRFGR PPTTLAEFSLNQYGKDYIDISNIKGFNVPMDFSPTTRGCRGVRCAADIVGQCPAKLKAPGGGCNDACTVFQTSEYCCTTGKCGP TEYSRFFKRLCPDAFSYVLDKPTTVTCPGSSNYRVTFCPTA
The term “Thaumatin A” refers to an oligopeptide or protein of the following amino acid sequence (SEQ ID NO: 3):
ATFEIVNRCSYTVWAAASKGDAALDAGGRQLNSGESWTINVEPGTNGGKIWARTDCYFDDSGSGICKTGDCGGLLRCKRFGR PPTTLAEFSLNQYGKDYIDISNIKGFNVPMDFSPTTRGCRGVRCAADIVGQCPAKLKAPGGGCNDACTVFQTSEYCCTTGKCGP TEYSRFFKRLCPDAFSYVLDKPTTVTCPGSSNYRVTFCPTA
The term “Thaumatin B” refers to an oligopeptide or protein of the following amino acid sequence (SEQ ID NO: 4):
ATFEIVNRCSYTVWAAASKGDAALDAGGRQLNSGESWTINVEPGTKGGKIWARTDCYFDDSGSGICKTGDCGGLLRCKRFGR PPTTLAEFSLNQYGKDYIDISNIKGFNVPMDFSPTTRGCRGVRCAADIVGQCPAKLKAPGGGCNDACTVFQTSEYCCTTGKCGP TEYSRFFKRLCPDAFSYVLDKPTTVTCPGSSNYRVTFCPTA
The Thaumatins may be used singly or two or more may be used jointly in the invention. For example, Thaumatin A and Thaumatin B may be used in combination. Alternatively, Thaumatin I and Thaumatin II may be used in combination. Where mixtures of two or more Thaumatins are used, any amounts or ratios with at least one Mogroside refer to the combination of the two or more Thaumatins, unless stated otherwise. However, as Thaumatin II has the most preferred organoleptic properties as a sweetener, Thaumatin II is preferred and the use of Thaumatin II as the sole Thaumatin is most preferred.
The term “Siratose” refers to a, preferably sweet, molecule with the structure:

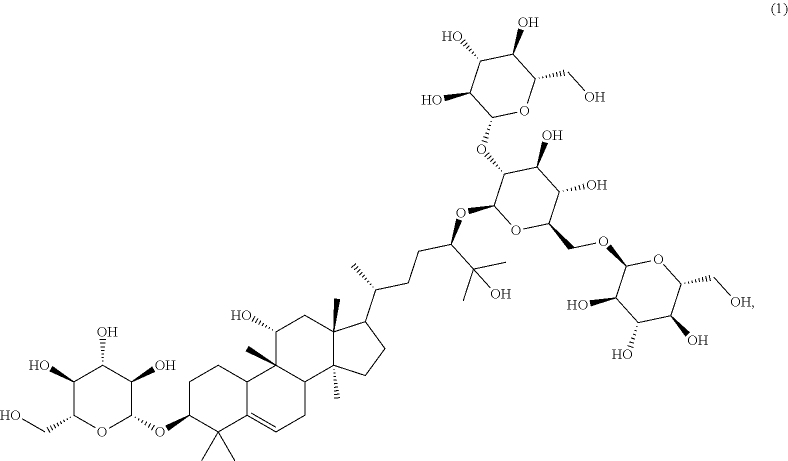 [SMILES: C[C@H](CC[C@@H](O[C@@H]1O[C@H](CO[C@H]2O[C@H](CO)[C@@H](O)[C@H](O)[C@H]2O)[C@@H](O)[C@H](O)[C@H]1O[C@H]1O[C@@H](CO)[C@H](O)[C@@H](O)[C@@H]1O)C(C)(C)O)C1CC[C@@]2(C)C3CC=C4C(CC[C@H](O[C@@H]5O[C@H](CO)[C@@H](O)[C@H](O)[C@H]5O)C4(C)C)[C@]3(C)[C@H](O)C[C@]12C]

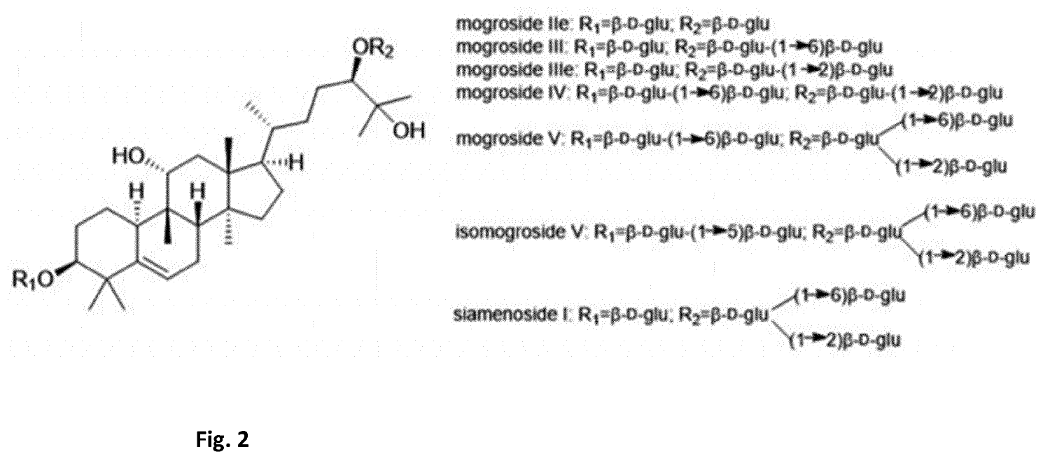

The term “Mogroside” refers to a sweet molecule with the structure shown in FIG. 2.

The term “temporal profile” of a composition, sugar or sweetener, as used herein, is a measure of the perceived sweetness intensity of said composition, sugar or sweetener over time. A desirable or advantageous temporal profile is one wherein sweetness is observed quickly and has a short linger similar to that of sucrose.

The term “sucrose equivalent value” or “SEV” as used herein refers to the sweetness equivalent of a sweetener related to the sweetness of sucrose. For example, a sweetener at an SEV value of 5 would have a sweetness similar to a 5% by weight solution of sucrose.

The term “low calorie” as used herein refers to a sweetener having 40 calories or fewer per reference amount customarily consumed (RACC) and per labeled serving. All amounts given in % by weight are quoted on a dry solids (ds) basis unless specifically stated otherwise.

According to an embodiment of the present invention, the sweetener or flavor modifying composition comprises Thaumatin, notably Thaumatin II, in an amount of about 1% to about 10%, and one or more Mogroside including Mog IIIe, Mog IV, Mog V, Mog VI, Siamenoside I, or Siratose in an amount of about 90% to about 99% by weight relative to the total weight of the composition. The thaumatin and mogroside portion of the sweetener or flavor modifying composition makes up between about 0.0001% (1 ppm) to about 0.1% (1000 ppm) by weight of the final food or beverage composition.

Preferably, the sweetener composition of the invention comprises Thaumatin, notably Thaumatin II, (preferably >90% purity) in an amount of about 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%, and a Mogroside including Mog IIIe, Mog IV, Mog V, Mog VI, Siamenoside I, or Siratose in an amount of about 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, or 99% by weight relative to the total weight of the composition. Preferably, the sweetener or flavor modifying composition would be used in the final food or beverage composition at about 1 ppm, 5 ppm, 10 ppm, 15, ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 150 ppm, 200 ppm, 250 ppm, 300 ppm, 350 ppm, 400 ppm, 450 ppm, 500 ppm, 550 ppm, 600 ppm, 650 ppm, 700 ppm, 750 ppm, 800 ppm, 850 ppm, 900 ppm, 950 ppm, or 1000 ppm by weight of the final food or beverage composition.

In a preferred embodiment of the present invention, the sweetener composition comprises Thaumatin, notably Thaumatin II, preferably of greater than 90% purity, in an amount of about 3% (preferably, about 1% to about 5%, or about 2% to about 4%), and combined Mogrosides including Mog IIIe, Mog IV, Mog V, Mog VI, Siamenoside I, and/or Siratose in an amount of about 97% (preferably, 95% to about 99%, or about 96% to about 98%) by weight relative to the total weight of the composition.

In an alternative embodiment, the sweetener composition comprises

Thaumatin, preferably Thaumatin II, preferably of greater than 90% purity, in an amount of about 7% (preferably, about 5% to about 10%, or about 6% to about 8%), and combined Mogrosides including Mog IIIe, Mog IV, Mog V, Mog VI, Siamenoside I, and/or Siratose in an amount of about 93% (preferably, 90% to about 95%, or about 92% to about 94%) by weight relative to the total weight of the composition.

Advantageously, the sweetener or flavor modifying composition comprises Thaumatin, preferably Thaumatin II, (preferably of a purity greater than 90%) in an amount of about 50% and one or more Mogrosides including Mog IIIe, Mog IV, Mog V, Mog VI, Siamenoside I, and/or Siratose in an amount of about 50% by percentage of added sweetness in terms of relative sugar equivalent value (SEV). In an alternative embodiment, the sweetener or flavor modifying composition comprises Thaumatin, preferably Thaumatin II, (preferably of a purity greater than 90%) in an amount of about 30% and one or more Mogrosides including Mog IIIe, Mog IV, Mog V, Mog VI, Siamenoside I, and/or Siratose in an amount of about 70% by percentage of added sweetness in terms of relative sugar equivalent value (SEV). In some embodiments, the sweetener composition may further comprise a sweet taste improving additive, a bulking agent, solvent, a flavoring agent, and/or a stabilizer.

A further aspect of the present invention provides a food product comprising the sweetener or flavor modifying composition of the invention. Non-limiting examples of a food product include a confectionary product, a dessert product such as yogurt, ice-cream, biscuits, and cakes, a cereal product, baked goods, frozen dairy products, meats, dairy products, condiments, snack bars, soups, dressings, mixes, prepared foods, baby foods, diet preparations, syrups, food coatings, dried fruit, sauces, gravies, jams/jellies, and the like, especially those which are reduced sugar or low sugar products. The food product may be an animal feed product. The food product of the invention may comprise a sweetener or flavor modifying composition as a coating or frosting formed on the surface of the product. A coating improves the flavor of the food product as well as its shelf life.

Another aspect of the invention provides a beverage product comprising a sweetener composition of the present invention. Non-limiting examples of a beverage product include a carbonated beverage, a non-carbonated beverage, fruit-flavored beverage, fruit-juice, tea, milk, coffee especially those which are reduced sugar or low sugar products. A further aspect of the present invention provides a table-top sweetener

comprising a sweetener composition according to the invention.

Another aspect of the present invention provides a bulking agent comprising a sweetener composition according to the invention.

Another aspect of the present invention provides a coating agent comprising a sweetener composition according to the invention.

Another aspect of the present invention provides a pharmaceutical product comprising a sweetener composition according to the invention.

Another aspect of the present invention provides a nutritional or sports product comprising a sweetener composition according to the invention.

Another aspect of the present invention provides a cosmetic product comprising a sweetener composition according to the invention.

It will be appreciated that the amount of a sweetener composition according to the invention present in a food product, a beverage product, a pharmaceutical product, a nutritional product, a sports product, or a cosmetic product, will depend upon the type and amount of sweetener present in the sweetener composition and the desired sweetness of the food or beverage product.

An alternative aspect of the present invention provides the use of a sweetener or flavor modifying composition according to the invention in a food product, a beverage product, a pharmaceutical product, a nutritional product, a sports product, or a cosmetic product, as a bulking agent or as a coating agent.

The sweetener or flavor modifying composition may be formulated in any physical form, for example, dissolved in a solvent such as water or glycol, as a syrup, in powder form, tablet form, as granules, in a solution or in any other suitable form including beverages and food products.

As outlined in the below examples, the sweetener or flavor modifying composition of the invention exhibits a sucrose equivalent value (SEV) greater than the predicted value based on its individual components. Therefore, the sweetener composition of the present invention displays sweetness synergy.

The following examples are exemplary only and is not intended to be limiting in any way.

### Example 1: Demonstration of Sweetness Synergy of the Composition of the Present Invention

Round table evaluations were performed with test panelists. Equal sweet 5 SEV concentrations in neutral pH water were made for Thaumatin II (purity greater than 90%), Mogroside V, and Siamenoside I. These equal sweet solutions were also mixed at 50% of Thaumatin II and 50% Mogroside V or Siamenoside I and compared to the individual components. The components of the test compositions are described in the below tables. The mixed compositions were calculated using the Beidler mixture equation for the sweeteners. The Beidler mixture equation for sweeteners is as follows:

$$SEV = \frac{conc \cdot Rmax}{conc + 1 / K}$$

The concentration of each component in the mixture in ppm is divided by SEV (c/R) and is plotted against concentration, c. The slope of the linear regression is the maximum SEV (Rₘₐₓ). The y-intercept of the linear regression multiplied by Rₘₐₓis the half-maximal sweetness concentration, 1/K. Rₘₐₓ and 1/K are the two parameters used in the Beidler equation. Mixtures were tasted in comparison to reference samples for the panelists to determine SEV values. References samples were 3%, 4%, 5%, 6%, 7%, and 8% sucrose in neutral pH water. The test samples were served in 2 ounce soufflé cups (approximately 60 ml) coded with 3-digit codes at room temperature. A two minute wait period between samples was enforced. Water and unsalted crackers was available for the panelists to clear their palates before and during testing. Results were collected for calculating approximate SEV level of each test sample.
The test products analyzed in this experiment are described below in Table 1.

### Product Information

TABLE 1
The mean sweetness intensity for Mogroside V, Siamenoside
I, Thaumatin II, and the combinations thereof.

| | Sweetness intensity |
|---|---|
| Composition | determined, SEV |
| 7 ppm Thaumatin II | 4.8 |
| 250 ppm Mogroside V | 5.3 |
| 250 ppm Siamenoside I | 6.0 |
| 3.5 ppm Thaumatin II/ | 5.8 |
| 125 ppm Mogroside V | |
| 3.5 ppm Thaumatin II/ | 6.4 |
| 125 ppm Siamenoside I | |

Surprisingly combinations of Thaumatin II and Mogroside V or Siamenoside I provided sweeter solutions than the individual components would suggest.

### Example 2: Qualitative Analysis of Individual Sweeteners and Mixtures Thereof

Round table evaluations were performed with 6 test panelists. Equal sweet 5 SEV concentrations in neutral pH water were made for Thaumatin II (purity greater than 90%), Mogroside V, and Siamenoside I. These equal sweet solutions were also mixed at 50% of Thaumatin II and 50% Mogroside V or Siamenoside I and compared to the individual components. All solutions were coded and blinded from panelists. Panelists were asked to provide a qualitative assessment on a scale of 1-10 of the quality of sweetness (10 being the highest quality of sweetness) and undesirable attributes (10 being most undesirable) of each solution using a 5% sucrose solution as a control.

TABLE 2
The mean taste quality of Thaumatin II, Mogroside
V, Siamenoside I, and the combination of thereof.

| | Quality of | | |
|---|---|---|---|
| | sweetness | Off taste | |
| | (higher | (lower | |
| Sample | better) | better) | Comments |
| 7 ppm Thaumatin II | 5.3 | 4.9 | Slow onset, |
| | | | sweet |
| | | | aftertaste |
| 250 ppm Mogroside V | 7.5 | 3.3 | Relatively |
| | | | clean |
| | | | sweetness |
| 250 ppm Siamenoside I | 7.8 | 2.6 | Relatively |
| | | | clean |
| | | | sweetness |
| 3.5 ppm Thaumatin II/ | 8 | 4.1 | Relatively |
| 250 ppm Mogroside V | | | clean |
| | | | sweetness, |
| | | | decreated |
| | | | sweet |
| | | | aftertaste |
| 3.5 ppm Thaumatin II/ | 8 | 3.5 | Relatively |
| 250 ppm Siamenoside I | | | clean |
| | | | sweetness, |
| | | | decreased |
| | | | sweet |
| | | | aftertaste |

Thaumatin II showed clean sweetness without any off taste. The sweetness onset was however slightly delayed, and sweet aftertaste lasted longer if compared with sucrose. In case of Mogroside V and Siamenoside, the sweetness onset was rapid, very mild off notes were detected. Surprisingly, mixtures of Thaumatin II and Mog V had high quality of sweetness and no pronounced off taste. Mixtures of Thaumatin II and Siamenoside I had even more pronounced improvements in sweet taste quality.

### Example 3: Analysis of Total Sweet Perception for Thaumatin II, Mogroside V and Their Mixture

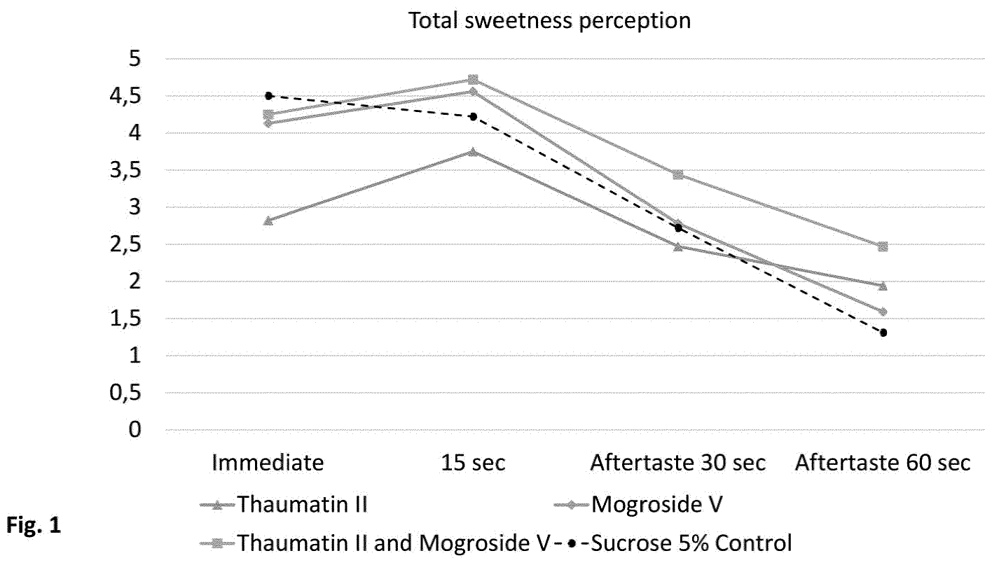

Recombinant Thaumatin II was purified from Nicotiana tabacum plants transgenic for ethanol-inducible replicon of Tobacco Mosaic Virus carrying Thaumatin II coding sequence. Thaumatin II expression was achieved by spraying plants with diluted ethanol solution. Thaumatin II protein was purified from plant biomass using column chromatography. The details of upstream and downstream processes are described in WO 2022/012926 A1.
7 ppm recombinant Thaumatin II and 250 ppm Mogroside V water solutions as well as both mixed together in the half-concentrations (3.5 ppm Thaumatin II/125 ppm Mogroside V) were analysed for the total sweet perception. 5% sucrose solution was used as a sweetness control. Flavor and aftertaste were assessed by 8 members of the trained sensory panel of Brisan Group (Orland Park, IL, USA) trained in the Spectrum descriptive methodology. The strength of each attribute was rated on the 15-point Spectrum Scale, where 0=none and 15=very strong. Products were evaluated at room temperature. Products were blinded with 3-digit codes and randomized. Breaks were given in between samples to reduce fatigue. All references were available to panelists to determine intensity scores. Flavour and aftertaste data was collected as individual data. All samples were expectorated. Samples were presented blind in a monadic sequential balanced design. Two replications of sample data were collected.
Panelists evaluated the total sweetness intensity immediately after starting the exposure to test solutions (0-5 seconds in the mouth) and at 15 seconds while the product was still in the mouth. Panelists also evaluated the aftertaste of products 30 seconds and 60 seconds after evaluating the product in mouth (after the expectoration).
To analyse the immediate sweet perception, panelists evaluated the sweet intensity immediately after taking a sip of the solution (evaluated within 0-5 seconds). The total sweetness of products was defined as a sum of the sweet basic taste and high intensity sweetener aromatic attribute. The sweet basic taste was defined as the taste on the tongue stimulated by sucrose and other sugars, such as fructose, glucose, etc. High intensity sweetener aromatic was defined as the light aromatic that is inherent with any non-sucrose sweetener, such as Aspartame, Ace-K, Reb A, Stevia, and Sucralose.
FIG. 1 summarizes the data on the total mean sweet perception for Thaumatin II, Mogroside V, and the combination thereof. The total sweetness intensity of Thaumatin II and Mogroside V blend was higher than suggested by individual ingredients, which is indicative for the sweetness synergy. In contrast to Thaumatin II, the blend has fast sweetness onset, thus blend has an improved sweetness quality.

### CONCLUSIONS

A sweetener or flavor modifying composition according to the present invention exhibits a significant sweetness synergy in addition to a substantial improvement in taste quality and concomitant reduction in undesirable organoleptic properties. Although the invention is illustrated and described herein with reference to specific embodiments, the invention is not intended to be limited to the details shown. Rather, various modifications may be made in the details within the scope and range of equivalents of the claims and without departing from the invention.

1. A sweetener or flavor modifying composition comprising Thaumatin II of greater than 90% purity and at least one Mogroside.

2. The sweetener or flavor modifying composition according to claim 1, whereas the Thaumatin II of greater than 90% purity is not isolated from Thamautococcus daniellii.

3. The sweetener or flavor modifying composition according to claim 1, whereas the Mogrosides is selected from one or more of Mog IIIe, Mog IV, Mog V, Mog VI, Siamenoside I, and/or Siratose.

4. The sweetener or flavor modifying composition according to claim 1, whereas the Thaumatin II is present in an amount of about 2% to about 5%, and the Mogroside is present in an amount of about 95% to about 98% by weight relative to the combined weight of the Thaumatin and mogroside components of the composition.

5. The sweetener or flavor modifying composition according to claim 1, , whereas the Thaumatin II is present in an amount of about 2% and the mogroside is present in an amount of about 98% by weight relative to the combined weight of the Thaumatin and mogroside components of the composition.

6. The sweetener or flavor modifying composition according to claim 1, whereas the Thaumatin II is present in an amount of about 2% and Mogroside V is present in an amount of about 98% by weight relative to the combined weight of the Thaumatin and Mogroside components of the composition.

7. The sweetener or flavor modifying composition according to claim 1, whereas the Thaumatin II is present in an amount of about 2% and Siamenoside I is present in an amount of about 98% by weight relative to the combined weight of the Thaumatin and Mogroside components of the composition.

8. A sweetener or flavor modifying composition comprising Thaumatin and at least one Mogroside.

9. The composition according to claim 8, wherein the Thaumatin is selected from Thaumatin I, Thaumatin II, Thaumatin A, and Thaumatin B, preferably the Thaumatin is Thaumatin II.

10. The sweetener or flavor modifying composition according to claim 8, wherein the Mogrosides is selected from one or more of Mog IIIe, Mog IV, Mog V, Mog VI, Siamenoside I, and/or Siratose.

11. The sweetener or flavor modifying composition according to claim 8, wherein the Thaumatin is present in an amount of about 2% to about 5%, and the Mogroside is present in an amount of about 95% to about 98% by weight relative to the combined weight of the Thaumatin and mogroside components of the composition.

12. The sweetener or flavor modifying composition according to claim 8, wherein the Thaumatin, preferably Thaumatin II, is present in an amount of about 2% and the mogroside is present in an amount of about 98% by weight relative to the combined weight of the Thaumatin and mogroside components of the composition.

13. The sweetener or flavor modifying composition according to claim 8, wherein the Thaumatin, preferably Thaumatin II, is present in an amount of about 2% and Mogroside V is present in an amount of about 98% by weight relative to the combined weight of the Thaumatin and Mogroside components of the composition.

14. The sweetener or flavor modifying composition according to claim 8, wherein the Thaumatin, preferably Thaumatin II, is present in an amount of about 2% and Siamenoside I is present in an amount of about 98% by weight relative to the combined weight of the Thaumatin and Mogroside components of the composition.

15. A method of manufacturing a reduced-calorie product comprising adding the sweetener composition according to claim 1 to a food product, a beverage product, a pharmaceutical product, a nutritional product, a sports product, or a cosmetic product.

16. A food, beverage, nutritional product, cosmetic product, or pharmaceutical product comprising adding the sweetener or flavor modifying composition of claim 1 at a use level between about 1 ppm and about 1000 ppm.

17. A food, beverage, nutritional product, cosmetic product, or pharmaceutical product comprising adding the sweetener or flavor modifying composition of claim 1 at a use level of about 500 ppm.

18. A food, beverage, nutritional product, cosmetic product, or pharmaceutical product comprising adding the sweetener or flavor modifying composition of claim 1 at t a use level of about 50 ppm.

19. A kit-of-parts comprising, as a first part, a Thaumatin, preferably Thaumatin II, or a composition comprising the Thaumatin, and, as a second part, one or more Mogroside or a composition comprising the one or more Mogroside.
